SEQUENCE LISTING (18 sequences)

SEQ ID NO: 1 - protein, 24 aa, HOMO SAPIENS
MAPRGFSCLLLLTSEIDLPVKRRA

SEQ ID NO: 2 - protein, 24 aa, ARTIFICIAL SEQUENCE
  ANALOG OF HUMAN  HUMANIN PEPTIDE
MAPRGFSCLLLLTGEIDLPVKRRA

SEQ ID NO: 3 - protein, 24 aa, ARTIFICIAL SEQUENCE
  ANALOG OF HUMAN HUMANIN PEPTIDE
MAPRGFSALLLLTSEIDLPVKRRA

SEQ ID NO: 4 - protein, 24 aa, ARTIFICIAL SEQUENCE
  ANALOG OF HUMAN HUMANIN PEPTIDE
MAPRGFSCLLLLTSEIDLPVKRRA

SEQ ID NO: 5 - protein, 24 aa, ARTIFICIAL SEQUENCE
  ANALOG OF HUMAN HUMANIN PEPTIDE
MAPAGASCLLLLTGEIDLPVKRRA

SEQ ID NO: 6 - protein, 24 aa, ARTIFICIAL SEQUENCE
  ANALOG OF HUMAN HUMANIN PEPTIDE
MAPAGASCLLLLTSEIDLPVKRRA

SEQ ID NO: 7 - protein, 17 aa, ARTIFICIAL SEQUENCE
  ANALOG OF HUMAN HUMANIN PEPTIDE
PAGASCLLLLTSEIDLP

SEQ ID NO: 8 - protein, 18 aa, ARTIFICIAL SEQUENCE
  ANALOG OF HUMAN HUMANIN PEPTIDE
PAGASRLLLLTGEIIDLP

SEQ ID NO: 9 - protein, 32 aa, ARTIFICIAL SEQUENCE
  ANALOG OF HUMAN HUMANIN PEPTIDE
EFLIVIKSMAPRGFSCLLLLTSEIDLPVKRRA

SEQ ID NO: 10 - protein, 32 aa, ARTIFICIAL SEQUENCE
  ANALOG OF HUMAN HUMANIN PEPTIDE
EFLIVIKSMAPRGFSALLLLTSEIDLPVKRRA

SEQ ID NO: 11 - protein, 32 aa, ARTIFICIAL SEQUENCE
  ANALOG OF HUMAN HUMANIN PEPTIDE
EFLIVIKSMAPRGFSCLLLLTGEIDLPVKRRA

SEQ ID NO: 12 - protein, 32 aa, ARTIFICIAL SEQUENCE
  ANALOG OF HUMAN HUMANIN PEPTIDE
EFLIVIKSMAPAGASCLLLLTGEIDLPVKRRA

SEQ ID NO: 13 - protein, 27 aa, ARTIFICIAL SEQUENCE
  ANALOG OF HUMAN HUMANIN PEPTIDE
SALLRSPIPAPAGASRLLLLTGEIDLP

SEQ ID NO: 14 - protein, 24 aa, ARTIFICIAL SEQUENCE
  ANALOG OF HUMAN HUMANIN PEPTIDE
MARRGFSCLLLSTTATDLPVKRRT

SEQ ID NO: 15 - protein, 24 aa, ARTIFICIAL SEQUENCE
  ANALOG OF HUMAN HUMANIN PEPTIDE
MAPRGASCLLLLTSEIDLPVKRRA

SEQ ID NO: 16 - protein, 24 aa, ARTIFICIAL SEQUENCE
  ANALOG OF HUMAN HUMANIN PEPTIDE
MAPRGASCLLLLTGEIDLPVKRRA

SEQ ID NO: 17 - protein, 24 aa, ARTIFICIAL SEQUENCE
  ANALOG OF HUMAN HUMANIN PEPTIDE
MAPRGASCLLLLTGEIDLPVARRA

SEQ ID NO: 18 - protein, 24 aa, ARTIFICIAL SEQUENCE
  ANALOG OF HUMAN HUMANIN PEPTIDE
MAKRGLNCLPHQVSEIDLSVQKRI

(19) United States
(12) Patent Application Publication
(10) Pub. No.: US 2013/0109635 A1
(43) Pub. Date: May 2, 2013

(54) Humanin Decreases Liver Fat and Visceral Fat Accumulation

(71) Applicant: Radhika Muzumdar, Mamaroneck, NY (US)

(72) Inventor: Radhika Muzumdar, Mamaroneck, NY (US)

(21) Appl. No.: 13/694,176

(22) Filed: Nov. 2, 2012

Related U.S. Application Data
(60) Provisional application No. 61/554,704, filed on Nov. 2, 2011.

(51) Int. Cl. A61K 38/17

(52) U.S. Cl. CPC A61K 38/1709
USPC 514/21.4; 530/325

(57) ABSTRACT

Methods are provided for reducing visceral fat or reducing accumulation of visceral fat in a subject, or of reducing fat in a liver or reducing accumulation of fat in a liver of a subject, comprising administering a humanin or an active analog of humanin to the subject. Methods are also provided of increasing hepatic triglyceride secretion in a subject or of increasing hepatic microsomal triglyceride transfer protein (MTTP) expression in a subject.

This application claims benefit of U.S. Provisional No. 61/554,704 filed Nov. 2, 2011, the contents of which are hereby incorporated by reference.

This invention was made with government support under grant numbers R-01 AG035114-02; K-08 AG 027462; and 3K08 AG027462-03S1, awarded by the National Institutes of Health. The government has certain rights in the invention.

Throughout this application various publications are referred to by number in parentheses. The disclosures of these publications are hereby incorporated by reference in their entirety into the subject application to more fully describe the art to which the subject invention pertains.

### BACKGROUND OF THE INVENTION

Obesity and visceral fat (VF) is reaching epidemic proportions in the USA and worldwide. An estimated 39.8 million American adults are obese and more than 57% of American adults are overweight (CDC figures). The prevalence rate is approximately 1 in 6. (WHO World Health Report, 2003). In addition, more than 60 percent of Americans aged 20 years and older are overweight.

Obesity is associated with many serious co-morbidities. Non-alcoholic fatty liver disease (NAFLD) and the extreme form, non-alcoholic steatohepatitis (NASH), are major health problems associated with obesity in the developed countries. Using proton NMR spectroscopy, the Dallas Heart Study (a population-based cohort study performed in an ethnically diverse community in the USA) reported that one in three adult Americans have steatosis. The finding indicates that over 70 million adult Americans suffer from NAFLD. NAFLD has also reached epidemic proportions among populations typically considered at “low risk” for this liver condition, with a prevalence in China and Japan of 15% and 14% respectively, among adults. In addition, in the USA, 6.4 million adults have NASH.

no specific treatment exists for visceral fat deposition and HAFLD or NASH. Current therapeutic approaches include life style changes, metformin, thiazolidinediones and ursodeoxycholic acids, and treatment typically targets risk factors that contribute to the increase in VF and liver disease.

Effective therapies for treating VF, NAFLD and NASH are still sought, and the current invention provides such treatments.

### SUMMARY OF THE INVENTION

A method is provided of reducing visceral fat or reducing accumulation of visceral fat in a subject, or of reducing fat in a liver or reducing accumulation of fat in a liver of a subject, comprising administering a humanin or an active analog of humanin to the subject in an amount and manner effective to reduce visceral fat or reducing accumulation of visceral fat, or reduce fat in a liver or accumulation of fat in a liver.

Also provided is a method of increasing hepatic triglyceride secretion in a subject or of increasing hepatic microsomal triglyceride transfer protein (MTTP) expression in a subject, comprising administering a humanin or an active analog of humanin to the subject in an amount and manner effective to increase hepatic triglyceride secretion or hepatic MTTP expression.

Also provided is a humanin analog for reducing visceral fat or reducing accumulation of visceral fat in a subject.

Also provided is a pharmaceutical composition for reducing visceral fat or reducing accumulation of visceral fat in a subject, or of reducing fat in a liver or reducing accumulation of fat in a liver of a subject, comprising a humanin or an active analog of humanin and a pharmaceutically acceptable carrier.

Also provided is a pharmaceutical composition for increasing hepatic triglyceride secretion in a subject or of increasing hepatic microsomal triglyceride transfer protein (MTTP) expression, comprising a humanin or an active analog of humanin and a pharmaceutically acceptable carrier.

Additional objects of the invention will be apparent from the description which follows.

### BRIEF DESCRIPTION OF THE DRAWINGS

FIG. 1: No difference in food intake between the HNG-treated (intraperitoneal administration) and control groups on high-fat diet (HFD) for 4 weeks.

FIG. 2: Animals treated with HNG gained significantly less weight than control treated animals over the study period.

FIG. 3: HNG treated animals gained significantly less VF compared to controls on HFD.

FIG. 4: Intracerebroventricular (ICV) HNG increases serum triglycerides in vivo in rats.

FIG. 5: ICV HNG increases triglyceride secretion from liver in vivo in mouse.

FIG. 6: ICV HNG increases rate of triglyceride secretion from liver in vivo in rats.

FIG. 7: Daily treatment with HNG for 4 weeks significantly decreased liver hepatic triglyceride content.

FIG. 8: Expression of MTTP was significantly higher in mice treated with HNG.

FIG. 9: Expression of ApoB was significantly higher in mice treated with HNG.

FIG. 10: Activity of MTTP was significantly higher in liver of HNG treated animals (p<0.01).

### DETAILED DESCRIPTION OF THE INVENTION

As used herein, “visceral fat” is also known as organ fat or intra-abdominal fat, and is located inside the abdominal cavity, packed in between organs (stomach, liver, intestines, kidneys, etc.). In obese subjects, excessive visceral fat is manifested. However, a person may be within a healthy weight range but still have excessive visceral fat around the internal organs.

Fatty liver, where fat accumulates in a subject's liver, is a reversible condition and occurs where large vacuoles of triglyceride fat accumulate in liver cells via the process of steatosis (i.e. abnormal retention of lipids within a cell). The associated fatty liver disease (FLD) may have an etiology not related to alcohol (non-alcoholic FLD (NAFLD)), or may be alcohol-related. A more severe form is non-alcoholic steatohepatitis (NASH).

In the development of FLD the hepatocytes present small fat vacuoles (liposomes) around the nucleus (microvesicular fatty change). In this stage liver cells are filled with multiple fat droplets that do not displace the centrally located nucleus. In the late stages, the size of the vacuoles increase pushing the nucleus to the periphery of the cell giving characteristic signet ring appearance (macrovesicular fatty change). These vesicles are well delineated and optically “empty” because fats dissolve during tissue processing. Large vacuoles may coalesce and produce fatty cysts which are irreversible lesions. Macrovesicular steatosis is the most common form and is typically associated with alcohol, diabetes, obesity and corticosteroids. Acute fatty liver of pregnancy and Reye's syndrome are examples of severe liver disease caused by microvesicular fatty change. The diagnosis of steatosis is made when fat in the liver exceeds 5-10% by weight. Severe fatty liver is sometimes accompanied by inflammation, a situation that is referred to as steatohepatitis. Progression to alcoholic steatohepatitis (ASH) or non-alcoholic steatohepatitis (NASH) depend on persistence or severity of inciting cause.

Fatty liver occurs when the amount of fat synthesized in the liver exceeds the amount of triglyceride secreted by it as VLDL. Accordingly, treatments that “increase” hepatic triglyceride secretion can be used to treat fatty liver. As used herein a treatment increases hepatic triglyceride secretion when it effects an increase in hepatic triglyceride secretion relative to the untreated state.

As used herein, humanin (HN) is a peptide having the sequence: MAPRGFSCLLLLTSEIDLPVKRRA (SEQ ID NO:1). An active humanin analog is a peptide or peptidomimetic based on humanin and having equivalent or improved activity thereof with regard hepatic triglyceride secretion described hereinbelow. In a preferred embodiment, the analog is a peptide. In an embodiment, the humanin or humanin analog is 17-50 amino acids in length. In a preferred embodiment the humanin or humanin analog is 20-25 amino acids in length. In a particularly preferred embodiment the humanin or humanin analog is 24 amino acids in length. Analogs of humanin are known in the art (e.g. see WO/2008/153788 A2). In a preferred embodiment, the active humanin analog is HNG, which has the sequence MAPRGFSCLLLLTGEIDLPVKRRA (SEQ ID NO:2). Analogs of humanin can also be created by substitution of conservative amino acids into humanin.

Other humanin analogs, including those containing D-amino acids, which can be used in the methods described include:

C8A-HN (HNA) MAPRGFSALLLLTSEIDLPVKRRA
D-Ser14-HN - MAPRGFSCLLLLTS*EIDLPVKRRA
AGA-HNG - MAPAGASCLLLLTGEIDLPVKRRA
AGA-(D-Ser14)- MAPAGASCLLLLTS*EIDLPVKRRA
HN AGA-(D-Ser14)- PAGASCLLLLTS*EIDLP
HN17 AGA-(C8R)- PAGASRLLLLTGEIIDLP
HNG17 EF-HN - EFLIVIKSMAPRGFSCLLLLTSEIDLPVKRRA
EF-HNA - EFLIVIKSMAPRGFSALLLLTSEIDLPVKRRA
EF-HNG - EFLIVIKSMAPRGFSCLLLLTGEIDLPVKRRA
EF-AGA-HNG - EFLIVIKSMAPAGASCLLLLTGEIDLPVKRRA
Colivelin - SALLRSPIPA-PAGASRLLLLTGEIDLP
P3R-HN - MARRGFSCLLLSTTATDLPVKRRT
F6A-HN - MAPRGASCLLLLTSEIDLPVKRRA
F6A-HNG - MAPRGASCLLLLTGEIDLPVKRRA
F6AK21A-HNG - MAPRGASCLLLLTGEIDLPVARRA
Z-HN - MAKRGLNCLPHQVSEIDLSVQKRI
S* indicates D-Serine
(SEQ ID NOS. 3-18, top to bottom,
respectively).

Active analogs of humanin are also described in US Patent Application Publication 2001/0039771 A1 and in US Patent Application Publication 2010/0204099 A1, the contents of each of which are hereby incorporated by reference.

A method is provided of reducing visceral fat or reducing accumulation of visceral fat in a subject, or of reducing fat in a liver or reducing accumulation of fat in a liver of a subject, comprising administering a humanin or an active analog of humanin to the subject effective to reduce visceral fat or reducing accumulation of visceral fat, or reduce fat in a liver or accumulation of fat in a liver.

In an embodiment, the method is of reducing visceral fat in a subject. In an embodiment, the method is of reducing accumulation of visceral fat in a subject. In an embodiment, the method is of reducing fat in a liver a subject. In an embodiment, the method is reducing accumulation of fat in a liver of a subject.

Also provided is a method of increasing hepatic triglyceride secretion in a subject or of increasing hepatic microsomal triglyceride transfer protein (MTTP) expression in a subject, comprising administering a humanin or an active analog of humanin to the subject effective to increase hepatic triglyceride secretion or hepatic MTTP expression.

In an embodiment, the method is of increasing hepatic triglyceride secretion in a subject. In an embodiment, the method is of increasing hepatic microsomal triglyceride transfer protein (MTTP) expression in a subject.

In an embodiment of the methods described hereinabove, the humanin or active analog of humanin is administered parenterally. In a preferred embodiment, the humanin or active analog of humanin is administered parenterally. In a most preferred embodiment the humanin or active analog of humanin is administered subcutaneously. In an embodiment, the humanin or active analog of humanin analog is administered into the central nervous system of the subject or in a manner effective to enter the central nervous system. In an embodiment, the humanin or active analog of humanin is administered intraperitoneally. In an embodiment, the humanin or active analog of humanin is administered via an implant in the subject. In an embodiment of the methods, the humanin or active analog of humanin is administered into the cerebrospinal fluid of the subject. In an embodiment of the methods, the humanin or active analog of humanin is administered via an implant in the subject. In a further embodiment, the implant comprises a polymer matrix. In an embodiment of the methods, the humanin or active analog of humanin is administered intraventricularly or intrathecally. In an embodiment of the methods, the humanin or active analog of humanin is administered intranasally. Direct administration can be effected by any method known in the art, including cannulation, catheterization, injection and via an implant, for example a subcutaneous implant.

In an embodiment of the methods, the active analog of humanin is administered. In a further embodiment, the humanin analog comprises SEQ ID NO:2. In an embodiment of the methods, the humanin is administered. In a further embodiment, the humanin comprises SEQ ID NO:1.

In an embodiment of the methods, the subject has non-alcoholic fatty liver disease (NAFLD) or non-alcoholic steatohepatitis (NASH). In an embodiment, the methods further comprise diagnosing the subject as suffering from non-alcoholic fatty liver disease or non-alcoholic steatohepatitis prior to administration of the humanin or humanin analog.

In an embodiment of the methods, the subject is not suffering from a neurodegenerative disorder. In an embodiment of the methods, the subject is diagnosed as obese prior to administration of the humanin or humanin analog. In a further embodiment, the method further comprises diagnosing the subject as obese. In an embodiment of the methods, the subject is overweight.

In an embodiment of the methods, it is the humanin analog which is administered, and the humanin analog comprises SEQ ID NO:2.

The humanin and humanin analogs described herein can be administered to the subject in a pharmaceutical composition comprising a pharmaceutically acceptable carrier. The pharmaceutically acceptable carrier used can depend on the route of administration. As used herein, a “pharmaceutically acceptable carrier” is a pharmaceutically acceptable solvent, a suspending vehicle, for delivering the instant agents to the animal or human subject. The carrier may be liquid or solid and is selected with the planned manner of administration in mind. Liposomes are also a pharmaceutically acceptable carrier. Pharmaceutically acceptable carriers are known in the art, and include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. In an embodiment the pharmaceutical carrier is acceptable for enteral or parenteral administration into the central nervous system of a mammal.

The inhibitors, active fragments, active analogs of fragments, and agents can be administered together or independently in admixtures with suitable pharmaceutical diluents, extenders, excipients, or carriers (collectively referred to herein as a pharmaceutically acceptable carrier) suitably selected with respect to the intended form of administration and as consistent with conventional pharmaceutical practices.

Techniques and compositions for making dosage forms useful in the invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Dosing can be any method or regime known in the art. For example, daily, twice daily, weekly, bi-weekly, monthly, as needed, and continuously. Implants are advantageous for continuous administration, but are not the only means of continuous administration.

In an embodiment the humanin or humanin analog is conjugated to an entity that improves the half-life and/or stability of the humanin or humanin analog. Non-limiting examples include PEG or derivatives thereof.

The peptide humanin and peptide humanin analogs can be synthesized by any technique known in the art including solid-phase synthesis, liquid-phase synthesis, and expression of appropriate encoding DNA in a host cell and recovery therefrom.

In accordance with the methods of the present invention, the subject is a mammal. Preferably, the subject is a human.

The methods disclosed herein are useful for treating an obese subject. An “obese” subject as used herein is characterized by the subject having a body mass index of 30.0 or greater (and thus includes the states of significant obesity, morbid obesity, super obesity, and super morbid obesity). In regard to gender, women with over 30% body fat are considered obese, and men with over 25% body fat are considered obese. The methods of disclosed herein are also applicable to treating an overweight subject, defined as a body mass index of the subject of from 25.0 to 29.9. The methods of disclosed herein are also applicable to treating a subject having excess VF or having a fatty liver.

Also provided is an active humanin analog for reducing visceral fat or reducing accumulation of visceral fat in a subject. In an embodiment, the humanin analog comprises SEQ ID NO:2.

Also provided is a pharmaceutical composition for reducing visceral fat or reducing accumulation of visceral fat in a subject, or of reducing fat in a liver or reducing accumulation of fat in a liver of a subject, comprising a humanin or an active analog of humanin and a pharmaceutically acceptable carrier.

Also provided is a pharmaceutical composition for increasing hepatic triglyceride secretion in a subject or of increasing hepatic microsomal triglyceride transfer protein (MTTP) expression, comprising a humanin or an active analog of humanin and a pharmaceutically acceptable carrier

Also provided is a method of identifying a candidate treatment for reducing visceral fat or reducing accumulation of visceral fat in a subject, or for reducing fat in a liver or reducing accumulation of fat in a liver of a subject, the method comprising a) modeling in silico the 3-dimensional form of the humanin analog comprising SEQ ID NO:2, b) testing in silico if a compound from a library of small molecule compounds mimics the modeled 3-dimensional form, and c) determining in vitro if the small molecule identified in b) is chemically stable, thereby identifying the candidate treatment.

In silico modeling of 3-D binding sites for rational drug design is known in the art. For example, see Computational Resources for Protein Modelling and Drug Discovery Applications, Infectious Disorders—Drug Targets (2009), 9, 557-562, B. Dhaliwal and Y. W. Chen, the contents of which are hereby incorporated by reference.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

### Introduction

The effect of humanin analog, HNG, on hepatic lipid fluxes was investigated. In the acute study, HNG was infused into third ventricle (ICV) of awake, unstressed and chronically catheterized Sprague-Dawley rats and C57BLK6 mice and studied plasma triglyceride (TG) levels following an injection of Tyloxapol (Triton WR1339, Sigma-Aldrich Corp, St. Louis, Mo.), an inhibitor of TG clearance. Groups of animals were also challenged with high fat diet (HFD) of 60% fat for one month and were treated with either daily injections of saline or of HNG (2 mg/kg). Body weight and Food intake were monitored twice a week. At the end of one month, liver fat was assessed both by histology and estimation of tissue triglycerides and plasma was collected. Total amount of visceral fat (VF) was determined and the specific depots were meticulously dissected and weighed. Tissue samples were snap-frozen for subsequent analysis.

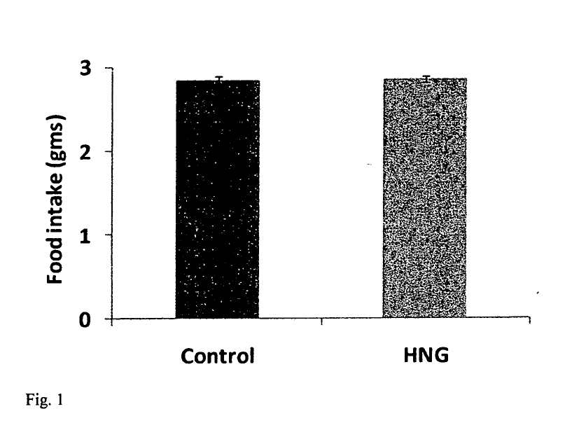

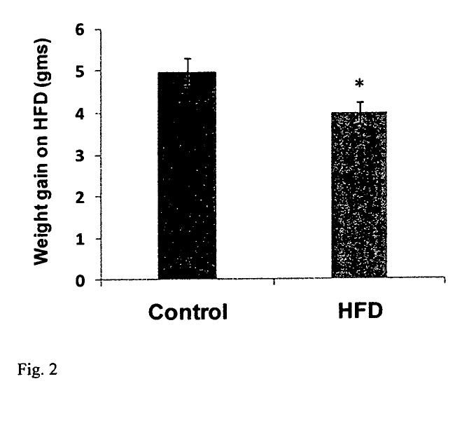

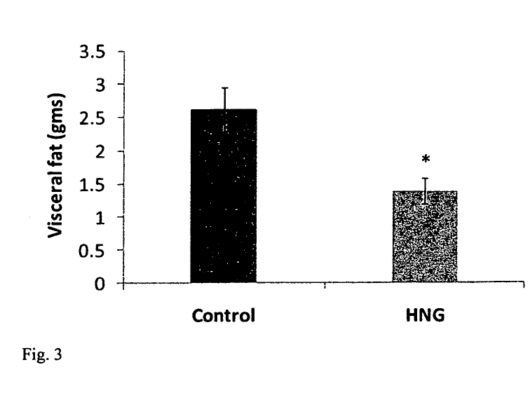

There was no difference in food intake between the HNG and control groups on HFD for 4 weeks (FIG. 1). Animals treated with HNG gained significantly less weight than control treated animals over the study period (FIG. 2). I-ING treated animals gained significantly less VF compared to controls on HFD (FIG. 3).

Effects of HN on hepatic lipid fluxes: Since insulin is a major regulator of hepatic lipid fluxes, and HN has important effects on hepatic insulin action, it was hypothesized based on the lack of observed weight increase in the above experiments that HN may regulate triglyceride secretion from the liver in vivo.

Acute effects of Humanin on liver fluxes: The effect of HN was studied in acute and chronic treatment models. In the acute study, HNG was infused into third ventricle (ICV) of awake, unstressed and chronically catheterized Sprague-Dawley rats and studied plasma TG levels following an injection of Tyloxapol (Triton WR1339, Sigma-Aldrich Corp, St. Louis, Mo.), an inhibitor of TG clearance.

Control (artificial CSF, aCSF, n=5) and HNG-infused rats (1 μg over 4 hours, n=5) were matched for age (3 mo), body weight (300±6.6 vs. 304±8.5 g), blood glucose levels (137±3.2 vs. 139±5.12 mg/dl) and serum triglycerides at baseline (0.2±0.007 vs. 0.28±0.08 mg/ml). Metabolic characteristics are summarized in Table 1.

| | Control | HNG | pValue |
|---|---|---|---|
| N | 5 | 5 | |
| Body weight (grams) | 300 ± 6.6 | 304 ± 8.5 | NS |
| Glucose (0 min) mg/dl | 137 ± 3.2 | 139 ± 5.12 | NS |
| Glucose (180 min) mg/dl | 139 ± 2.78 | 147 ± 8.09 | NS |
| Insulin (0 min) | 0.78 ± 0.1 | 0.81 ± 0.05 | NS |
| Insulin (180 min) | 0.59 ± 0.1 | 0.76 ± 0.09 | NS |
| Basal Triglyceride (mg/ml) | 0.2 ± 0.007 | 0.28 ± 0.08 | NS |

After an initial stabilization phase of 60 min., all animals received a bolus injection of tyloxapol (600 mg/kg) at 60 min. and HNG or aCSF (depending on the group) was infused ICV for 120 min. Blood samples were drawn for TG measurements just prior to tyloxapol administration and every 30 min. thereafter for 120 min.

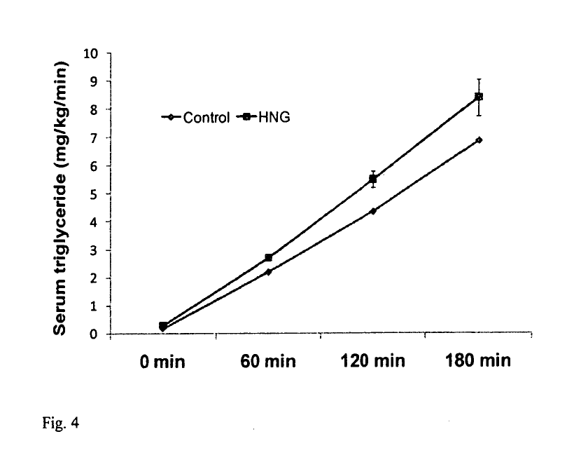

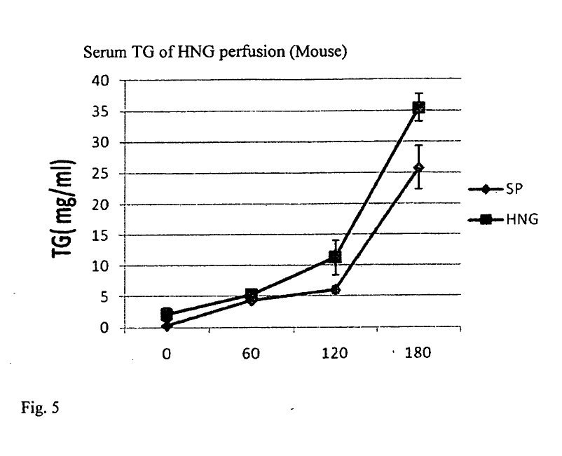

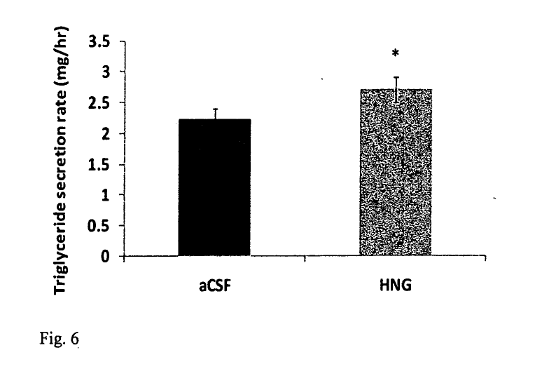

With infusion of tyloxapol, there was a steady increase in plasma triglyceride levels in both groups from inhibition of TG clearance. In rats, serum TG levels at 120 min were significantly higher in HNG as compared to control (HNG=5.5±0.29 vs. aCSF=4.33±0.33 mg/ml, p<0.05, FIG. 4). Similar results were also observed in mice (FIG. 5). The rate of TG secretion was calculated in the animals based on plasma triglyceride levels at 0 and 120 min time points using the following equation: secretion rate of TG/hr mg/min=(TG at 120 min−TG at 0 min)/number of hours. The rate of TG secretion in HNG was significantly higher (HNG=2.7±0.2 aCSF=2.23±0.16 mg/hr, p<0.05, FIG. 6) compared to controls.

Chronic effects of Humanin on liver lipid content: Groups of animals were challenged with high fat diet (HFD-60% fat) for one month and were treated with either daily injections of saline or HNG (2 mg/kg). At the end of one month, liver fat were assessed both by histology and estimation of tissue triglycerides.

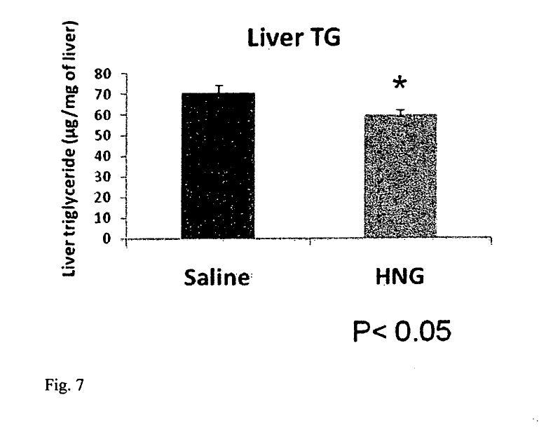

In the HFD group, the liver fat in the HNG treated group was significantly lower (HNG=59.78±2.24 vs. aCSF=70.83±3.74 μg/mg of tissue, p<0.05, FIG. 7). There was a significant correlation between liver TG measurements and estimation of liver fat by histological analysis (r=0.77). Serum TG tended to be higher in HNG treated animals at the end of 4 weeks and free fatty acids were not significantly different between the groups (data not shown).

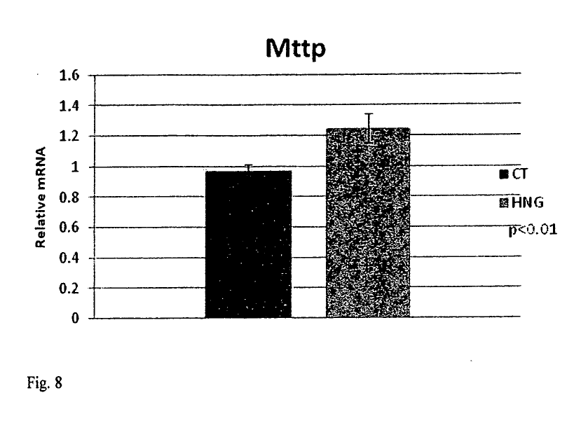

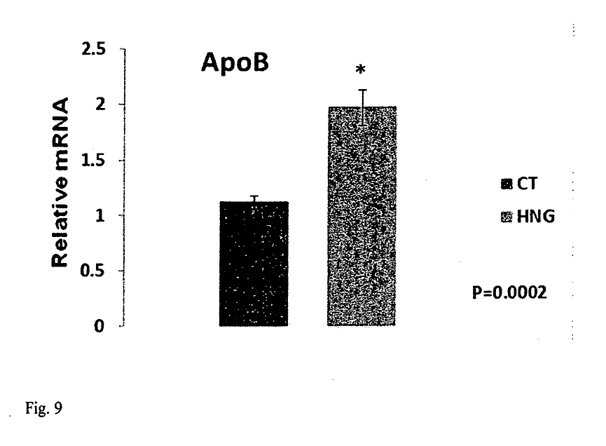

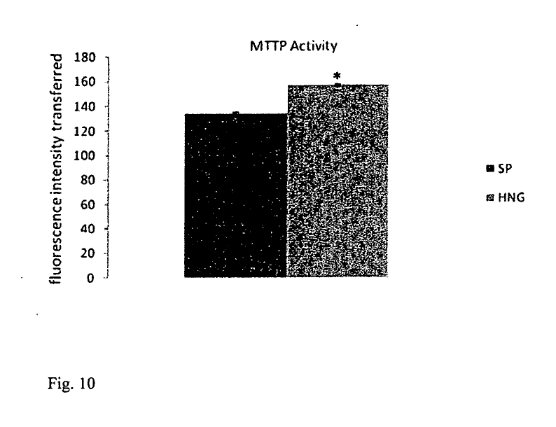

To understand the mechanisms through which Humanin decreases liver lipid content, representative genes involved in lipid synthesis (SREBP-1, FAS), lipid oxidation (CPT1b) and lipid packaging and export from liver were studied. There were no differences in the expression of FAS, SREBP-1 or CPT1b between control and HN treated groups (data not shown). The expression of Microsomal triglyceride transport protein (MTTP, FIG. 8) and Apo B (FIG. 9), however, were significantly higher in the livers of HNG-treated HFD animals. The role of MTTP was confirmed by studying enzyme activity. MTTP activity (Roar Biomedical, NY, NY) was significantly higher in the animals treated with HNG compared to controls (FIG. 10).

For the first time it is demonstrated that a humanin analog, HNG, decreases VF accumulation on HFD and potently affects hepatic triglyceride secretion in vivo. HNG decreases fat accumulation in liver on HFD through increases in expression and activity of MTTP, a key rate-limiting enzyme in triglyceride packaging and secretion from the liver.

What is claimed is:

1. A method of reducing visceral fat, or of reducing accumulation of visceral fat, or of reducing fat in a liver, or of reducing accumulation of fat in a liver, of a subject comprising administering a humanin or an active analog of humanin to the subject effective to reduce visceral fat, or reduce accumulation of visceral fat, or reduce fat in a liver or reduce accumulation of fat in a liver.

2. A method of increasing hepatic triglyceride secretion in a subject or of increasing hepatic microsomal triglyceride transfer protein (MTTP) expression in a subject, comprising administering a humanin or an active analog of humanin to the subject effective to increase hepatic triglyceride secretion or hepatic MTTP expression.

3. The method of claim 1 or 2, wherein the humanin or active analog of humanin is administered parenterally.

4. The method of any of claims 1-3, wherein the humanin or active analog of humanin is administered subcutaneously.

5. The method of any of claims 1-3, wherein the humanin or active analog of humanin analog is administered into the central nervous system of the subject or in a manner effective to enter the central nervous system.

6. The method of any of claims 1-5, wherein the humanin or active analog of humanin is administered intraperitoneally.

7. The method of any of claims 1-6, wherein the humanin or active analog of humanin is administered via an implant in the subject.

8. The method of claim 7, wherein the implant comprises a polymer matrix.

9. The method of any of claim 1, 5 or 7, wherein the humanin or active analog of humanin is administered intraventricularly or intrathecally.

10. The method of any of claims 1-4, wherein the humanin or active analog of humanin is administered intranasally.

11. The method of any of claims 1-10 wherein the active analog of humanin is administered and comprises SEQ ID NO:2.

12. The method of any of claims 1-11, wherein the subject has non-alcoholic fatty liver disease or non-alcoholic steatohepatitis.

13. The method of claim 12, further comprising diagnosing the subject as suffering from non-alcoholic fatty liver disease or non-alcoholic steatohepatitis prior to administration of the humanin or humanin analog.

14. The method of any of claims 1-13, wherein the subject is not suffering from a neurodegenerative disorder.

15. The method of any of claims 1-14, wherein the subject is obese prior to administration of the humanin or humanin analog.

16. The method of claim 12, further comprising diagnosing the subject as obese prior to administration.

17. The method of any of claims 1-16, wherein the subject is overweight.

18. A humanin analog for reducing liver fat or visceral fat, or for reducing accumulation of visceral fat or for increasing hepatic secretion in a subject.

19. The humanin analog of claim 18, wherein the humanin analog comprises SEQ ID NO:2.

20. A pharmaceutical composition for reducing liver fat or visceral fat, or for reducing accumulation of visceral fat in a subject, or of reducing fat in a liver or reducing accumulation of fat in a liver of a subject, comprising a humanin or an active analog of humanin and a pharmaceutically acceptable carrier.

21. A pharmaceutical composition for increasing hepatic triglyceride secretion in a subject or of increasing hepatic microsomal triglyceride transfer protein (MTTP) expression, comprising a humanin or an active analog of humanin and a pharmaceutically acceptable carrier.
